(19) United States
(12) Patent Application Publication
(10) Pub. No.: US 2007/0197899 A1
(43) Pub. Date: Aug. 23, 2007

(54) Apparatus and method for magnetic navigation using boost magnets

(76) Inventors: Rogers C. Ritter, Charlottesville, VA (US); Roger N. Hastings, Maple Grove, MN (US)

Correspondence Address: Bryan K. Wheelock, 7700 Bonhomme, Suite 400, St. Louis, MO, 63105, US

(21) Appl. No.: 11/653,780

(22) Filed: Jan. 16, 2007

Related U.S. Application Data
(60) Provisional application No. 60/759,597, filed on Jan. 17, 2006.

(51) Int. Cl. A61B 5/05

(52) U.S. Cl. 600/407; 600/411; 600/424

(57) ABSTRACT

A method of and system for navigating a medical device in a subject. The device has a magnet in a tip of the device and is navigable in the subject using source magnets positioned outside the subject. A source magnet magnetic field is used to navigate the device tip to a point in the subject. A boost magnetic moment is created by boost coils and is added to a permanent tip magnet moment to increase the torque applied by the externally generated magnetic field to the device tip. At least one boost magnet is used to apply the boost magnetic field. This method also makes it possible to design magnetic navigation systems with reduced size and cost.

### CROSS-REFERENCE TO RELATED APPLICATIONS

This application claims the benefit of U.S. Provisional Patent Application Ser. No. 60/759,597, filed Jan. 17, 2006, the entire disclosure of which is incorporated herein by reference.

### FIELD

The present invention relates to magnetically navigating medical devices and more particularly to using boost magnets to facilitate navigation of medical devices, including devices with magnetically responsive tips.

### BACKGROUND

During interventional surgery, devices such as catheters and guide wires may be navigated along complex pathways, e.g., through the blood vessels of a subject, to anatomical sites deep within the subject's body for diagnostic or therapeutic purposed. In some recently developed medical navigation systems, medical devices can be steered within the subject's anatomy by external application of a suitable magnetic field to the subject. In such systems, one or more magnets are present in the vicinity of a distal portion of the medical device. One or more source magnets located near the subject may be used to externally generate the magnetic field applied to an operating region. A catheter or guide wire can be steered by altering the direction of the applied magnetic field.

Coil tipped catheters have been described for magnetic navigation in a magnetic field with a large dominant component of fixed direction such as that generated by a Magnetic Resonance Imaging (MRI) system. U.S. Pat. No. 6,834,201, “Catheter Navigation within an MR Imaging device,” and cited patents within, describe devices and navigation methods in which it is not possible to control the torque that would be applied by the magnetic field to a permanent magnet catheter tip. The present disclosure describes the use of coils and permanent magnets on a catheter tip in conjunction with a controlling variable source field.

Current magnetic navigation systems provide very large source magnet fields so that enough torque can be achieved to guide a small device tip magnet by orienting it and bending the device at or near its distal end against its mechanical restraining torque. The symmetry of a source field is determined by the number of source magnets and their individual designs and shapes. Source magnet configurations generally cannot generate an optimal magnetic field in all directions. Subject and imaging access constraints generally limit the size of the source magnet and associated mechanism and make the use of additional source magnets difficult. In any case it is generally not possible to provide relatively uniform fields over reasonable operating regions and over all field directions without “overdesigning” the source magnets. That is, the source magnet sizes and shapes that provide sufficient field in the “weakest” field magnitude-field direction combination are oversized for the majority of directions.

### SUMMARY

One aspect of the present invention is directed to a method of controlling a medical device in a subject. The device has at least one magnetically responsive element at or near the distal tip and is navigable in the subject using at least one source magnet positioned outside the subject. A source magnet magnetic field is used to navigate the device tip in a first direction. A boost magnetic moment is applied to a tip magnet moment to increase the total tip moment in the appropriate direction at the appropriate time to overcome the relative weakness of the source field in such particular situations where the externally applied field and tip magnet moment might not suffice to induce the required tip orientation against the device restraining mechanical torque. At least one boost magnet is used to generate the boost magnetic moment. The boost magnet on the catheter tip in one embodiment of this invention can comprise one or more coils or sets of coils, and preferably three coils or sets of coils, able to increase the moment by at least 10 percent, and preferably by at least 30 percent, in any direction relative to the tip magnet axis. Such a design can therefore be expected to allow an equivalent reduction in the strength of the source magnets, either 10 percent or 30 percent, respectively. It is also to be expected that the additional degrees of freedom provided by these adjustable added moments will further enable optimization of the source magnet designs in several ways. These might include differences in shape of the source magnets; in turn or additionally they might include novel arrangements of internal field direction as used for focusing in several permanent source magnet designs.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a schematic diagram of a system for navigating a medical device in accordance with one embodiment of the invention;

FIG. 2 is a perspective view of a source magnet showing lines of equal field intensity;

FIG. 3 is a perspective view of two source magnets;

FIG. 4 is a perspective view of a catheter tip magnet with three booster coils providing three orthogonal directions of boost moment;

FIG. 5 is a perspective view of a guide wire tip magnet having two pairs of boost coils in accordance with one implementation of the invention;

FIG. 6 presents diagrams 6-A to 6-C for one embodiment of a tip magnet being turned in a magnetic field in accordance with one embodiment and application of the invention;

FIG. 7 presents diagrams 7-A to 7-C for a second embodiment of a tip magnet being turned in a magnetic field in accordance with a second embodiment and application of the invention;

FIG. 8-A is a plan view of a pancake boost coil in accordance with one embodiment of the invention;

FIG. 8-B is an side elevation view of a pancake boost coil in accordance with one embodiment of the invention;

FIG. 9 is a plan view of a boost coil in accordance with one embodiment of the invention.

FIG. 10 is a flow diagram for a system design in accordance with one aspect of the invention;

FIG. 11 is a schematic view illustrating the application of one embodiment of the current invention to a sheath for specific cardiac applications;

FIG. 12 is a schematic diagram illustrating one embodiment of the current invention in the form of a catheter with several boost magnets at various locations along the device; and

FIG. 13 is a flow diagram for a navigation method using boost moments according to the present invention.

Corresponding reference numerals indicate corresponding parts throughout the several views of the drawings.

### DETAILED DESCRIPTION OF THE INVENTION

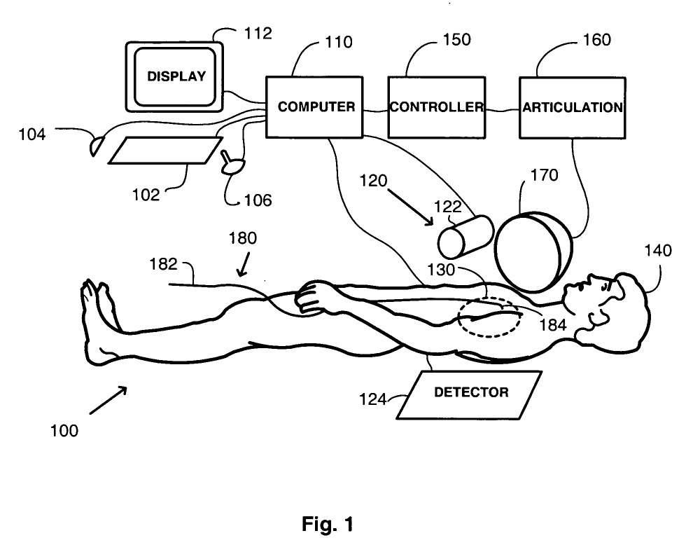

One exemplary embodiment of a system for navigating a medical device is indicated generally in FIG. 1 by reference number 100. A physician may use a keyboard 102, mouse 104, joystick 106, and/or other device to input instructions to a computer 110. The physician may also use a display 112 connected with the computer 110 to monitor navigation and to operate the system 100. An imaging apparatus 120 processes signals from the computer 110, acquires image data for an operating region 130 of a subject 140, and displays the corresponding images on the display 112. As an example, the imaging apparatus 120 may comprise an x-ray tube 122 and an x-ray detector 124 for the acquisition of radiographic or fluoroscopic images.

A controller 150 connected with the computer 110 controls an articulation mechanism 160 that translates and/or rotates one or more source magnet(s) 170. The source magnet(s) 170 create a magnetic field of specific magnitude and orientation in the subject operating region 130 to control the orientation of a medical device 180 having a proximal end 182 and a distal end 184. The distal end 184 comprises a magnetically responsive element at the tip 186 (not shown in FIG. 1). The source magnet(s) 170 may include permanent magnet(s) and/or electromagnet(s).

The medical device 180 may be, for example, a catheter, guide wire, sheath, endoscope, or other device that the physician wishes to navigate in the subject's body. The magnetically responsive elements at the tip 186 may comprise one or more permanent magnets and boost coils (not shown in FIG. 1) responsive to a magnetic field and/or gradient from the source magnet(s) 170. It should be noted that although several configurations are described in which a medical device includes one magnetically responsive element, configurations also are contemplated in which a device tip includes more than one magnetically responsive element; further, configurations are also contemplated in which a device comprises several sets of boost magnets at various locations along the length of the device.

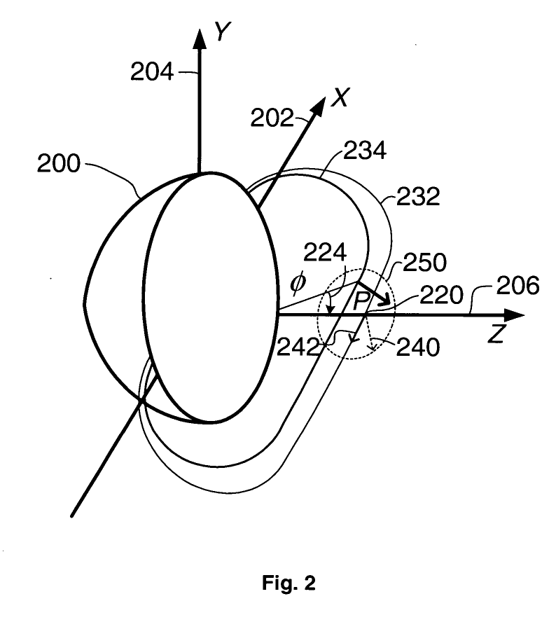

Magnetic moments immersed in a varying field are subject to forces dependent upon the field gradient; a magnetic moments subjected to a uniform field at an angle with respect to the moment is subject to a torque that tends to align the moment with the field; thus it is generally desirable in magnetic navigation to have a constant field strength applied at the catheter tip; the field direction being chosen to orient the catheter tip in a desired direction. Generally, it has been observed that magnet field sources used for magnetically navigating medical devices may be associated with one or more “difficult directions” in which to provide a guiding source field. For example, FIG. 2 schematically shows a plot of the lines of constant field strength in a given plane about a permanent magnet field source 200; field directions are shown by arrows. Although there is gross symmetry about a plane (X, Z) shown by the axes 202 and 206 in this particular magnet, it can be seen that when the magnet is pivoted to change the field direction imposed at point P 220 the magnitude of the field at that point will be increased. After pivoting the magnet by an angle Φ 224 with respect to Y axis 204, the point P will have moved from line 232 of constant field magnitude B₁ to line 234 of constant and greater field magnitude B₂. As a result of the magnet pivoting, point P will then lie on a line of greater field strength and will be subject to a field in the desired direction as indicated by arrow 240. It is apparent in this example that a magnet tip to be directed in the original field direction 242 at point P will experience a weaker field than in the second field direction. Therefore a set of boost coils on the tip could be used to change the tip moment to compensate for that difference in source field strength.

In general there may be more source magnets and the combinations of their fields at a given operating point P in a given operating region will require a set of boost coils capable of assisting the tip moment in almost any direction. The minimum set size required to boost the tip moment in any direction is three; preferably their axes would form an orthogonal set.

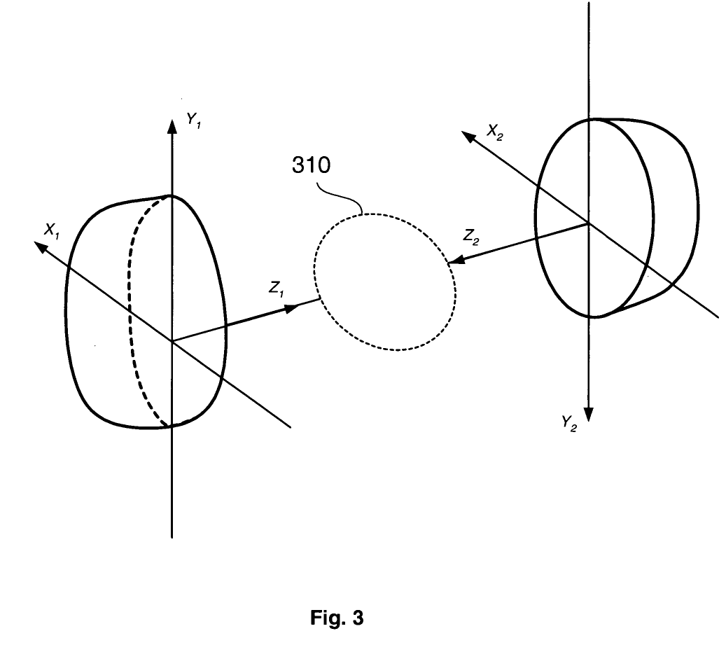

Lines of constant field strength in FIG. 2 illustrate the variation of field strength throughout a small region 250 in which a catheter tip might be navigated, an “operating region.” The variation in field strength is significant throughout this region. When such a magnet is combined with a similar one placed a distance away as in FIG. 3, the field variations throughout the operating region 310 can be reduced in general, but significant variations still remain. When two such source magnets are articulated appropriately the sum of their individual fields provides a roughly uniform field within the operating region; and this remains the case for most desired directions. But for the resultant field to remain constant throughout the complete range of application angles it may be necessary to pull away one or both of these source magnets (for example to maintain the magnets outside a subject's safety zone). In general, it might not be possible to correct for the field magnitude variations induced by motion of the first magnet by a compensating motion of the second magnet, signifying that the possible combinatory field had variation in strength, that it had strong regions and weak regions. The application of appropriate boost moment directions and magnitudes at the catheter tip, which would otherwise have a constant moment, allows compensating for the torque magnitude variations that would otherwise result from applying to the tip a field of varying magnitude. And in particular, when the appropriately changed tip moments would boost up the weakest field directions, the source magnets can thereby be made smaller.

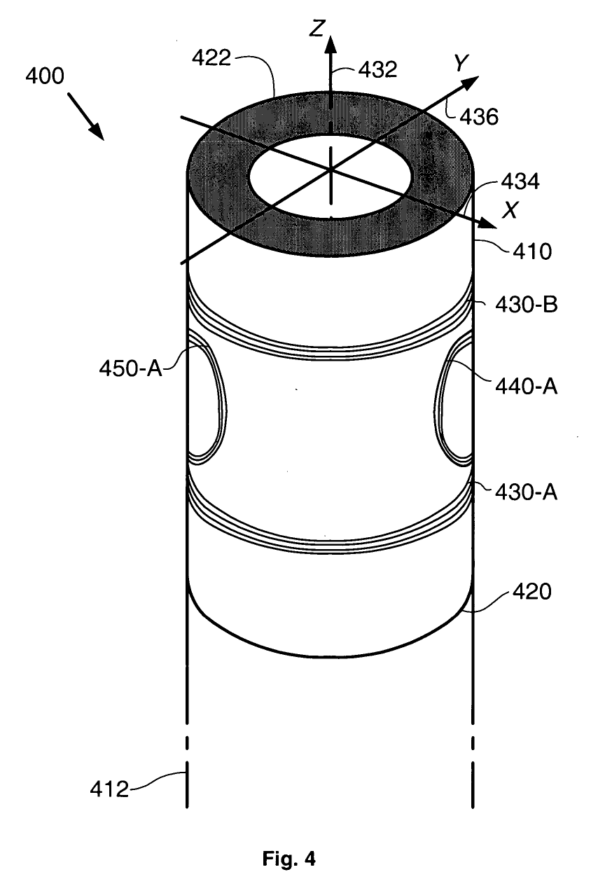

FIG. 4 shows a perspective view of an embodiment of a catheter according to the principles of this invention, generally indicated by reference number 400. The distal end 410 of catheter 412 comprises a tip magnet 420 and three pairs of coils suitable to provide a boost moment in any direction. The pair of coils (only a few wiring loops shown) 430-A and 430-B allows generation of an additional magnetic moment along Z axis 432. Two additional pairs of coils are mounted on the device wall along two orthogonal directions X, 434, and Y, 436. Only one of the two coils for each pair is shown schematically in the perspective view, respectively coil 440-A for generation of a moment along axis 434, and coil 450-A for generation of a moment along axis 436. In this embodiment, the tip magnet is made of a permanent or permeable magnetic material shaped in the form of a hollow cylinder 422 to allow insertion of the catheter over a guide wire (not shown).

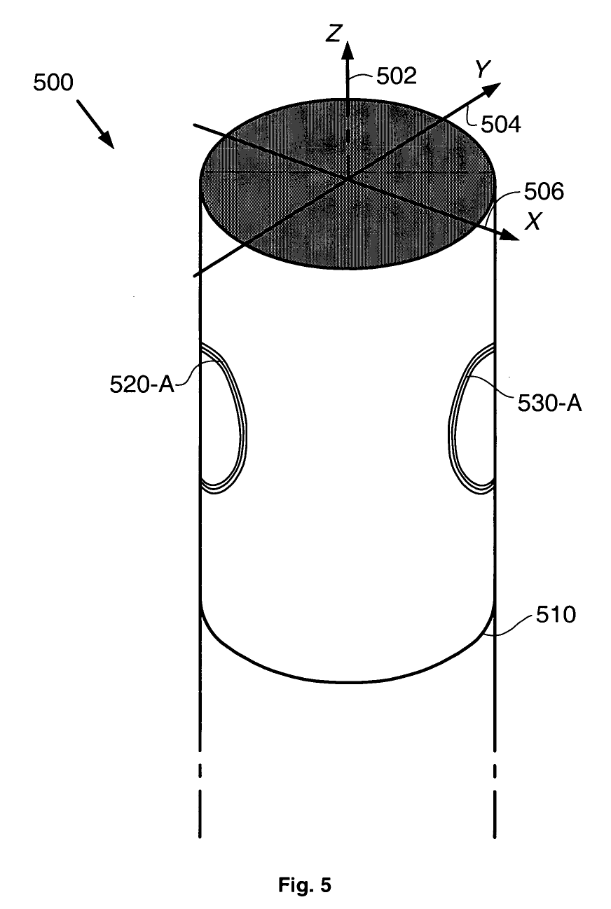

An exemplary embodiment of a magnet suitable for use in the tip of a guide wire or other medical device is indicated generally in FIG. 5 by reference number 500. A tip magnet 510 is generally cylindrical and made from a strong permanent magnetic material such as neodymium-iron-boron (NdFeB), although other suitable shapes and/or magnetic materials could be provided in other embodiments. The tip magnet 510 is magnetized along a longitudinal axis 502. Two pairs 520 A-B and 530 A-B of boost coils are attached to the magnet 510. The pairs 520 and 530 are aligned respectively with transverse axes 504 and 506 of the tip magnet 510 to provide a boost magnetic moment perpendicular to the longitudinal axis 502. Although two pairs of coils are described in the present exemplary embodiment, in other embodiments a single coil in each of the transverse axes 504 and 506 could provide the same or similar functionality as the coil pairs 520 and 530.

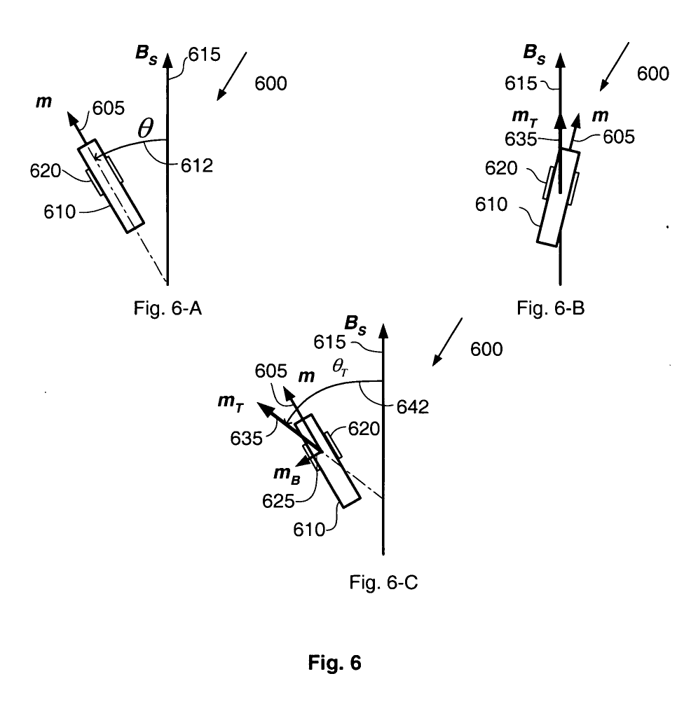

The tip magnet 510 may be positioned in a subject operating region, for example, as indicated generally in FIGS. 6-A to 6-C by reference number 600. A magnetic moment m 605 of the tip magnet 610 is oriented at a lead angle θ 612 of about 30 degrees relative to a navigating magnetic field \vec{B}s 615 in the situation shown. A lead angle is necessary in order to turn a catheter tip against its mechanical stiffness because the magnitude of the torque F applied to the moment by the field is proportional to the sine of the lead angle, a shown by the equation: r=mBₛ sin θ. Where it is desired to turn the tip magnet 610 to a position as shown in FIG. 6-B, the application of currents of appropriate polarity to the boost coils 620 create an adjustable moment mB 625 that interacts with the main source field \vec{B}s just as moment m does, see FIG. 6-C. The magnetic tip moment m and the boost moment m_{B} add vectorially resulting in a total moment m_{T} 635 of both increased magnitude and increased lead angle θ_{T} 642. Both the increased magnitude and lead angle contribute to increase the torque applied to the tip 610, which tends to align the total magnetic moment m_{T} with the applied field. In FIG. 6-C, the boost coils 620 are shown for illustration as significantly raised on the tip surface, although in practical implementations this need not be the case.

The coil pairs 520 and 530 shown in FIG. 5 make it possible to provide a boost moment in a direction anywhere in a plane perpendicular to the tip moment m. In FIG. 6 the boost coils 620 provide a boost magnetic moment 625 at an angle of 90 degrees relative to the magnetic moment m 605 of the tip magnet 610. In conjunction with a leading source magnetic field \vec{B}s, the resulting increased total moment MT can pull the tip magnet 610 towards an orientation parallel to the system source field \vec{B}s as shown in FIG. 6-B. If necessary, the currents provided to the boost coils 620 can be modulated during the tip reorientation to provide improved final alignment; FIG. 6-B shows the final alignment that would be obtained by maintaining the boost moment throughout the turn.

Where, for example, the system 100 of FIG. 1 is used to navigate a medical device tip including the magnet 610, the computer 110 may determine a boost moment direction and strength based on a target value for the lead angle θ_{T} and resulting total moment magnitude m_{T}=∥m_{T}∥ needed to counter the catheter restoring torque which will vary with the angle of its bending. Torque is applied to the magnet 610 in accordance with the relationship Γ=m_{T}B sin θ_{T}. This is the magnetic torque which must overcome the resisting mechanical torque associated with the guide wire or catheter stiffness.

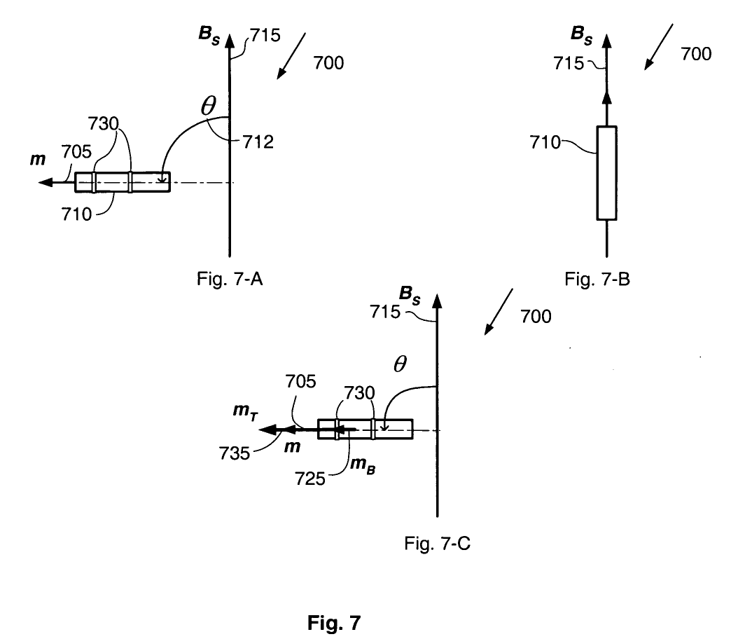

It is also the case that one or more boost coils can be wound around the tip magnet to increase its moment in the tip magnet moment direction. This is useful because in some cases the weakest direction of the source field may occur along an axis perpendicular to the tip magnet axis, and simply increasing the axial moment may be most effective. This situation is described in FIG. 7, where the device magnetic tip immersed in an orthogonal magnetic field is generally indicated by reference number 700. FIG. 7-A describes the relative geometry of the catheter or guide wire tip 710 with respect to the field \vec{B}s 715 applied in the direction to which it is desired to orient the tip 710. FIG. 7-B illustrates the desired result. In FIG. 7-C it is seen that by applying a current to a pair of booster coils 730, a boost magnetic moment m_{B} 725 is generated that is parallel with the tip moment m 705. In this situation, the resulting moment m_{T} 735 is parallel with the moment m 705 but its magnitude is larger by m_{B}, an amount that in specific situations will increase the applied torque sufficiently to bring about the tip alignment with the applied field. In general the need for boost can occur in any direction, and the computer can know from the source magnet system properties and the medical device properties what moment size and direction changes can be used to most efficiently overcome the lack of torque occasioned by the weakened source field at a particular location and in a particular direction.

It may be thought that the coils 430-A and 430-B could be redundant, capable only of boosting the field in the direction of the existing tip moment along the catheter axis. It is the case, however, that some combinations of source magnetic field configurations and given desired torque direction will actually require a “negative boost”, that is with longitudinal boost field opposing the existing tip moment field direction, in order to optimally (that is with minimal required transverse boost) turn the tip in a certain direction with large transverse component.

The heat dissipated in a catheter tip coil can be of concern. In U.S. Pat. No. 6,834,201 methods of pulsing the current to the coils and of removing heat by a heat exchange medium are described. In the present invention current may be applied, e.g., pulsed, to the boost coils 620 for only a portion of the time during which the tip magnet 610 is moved to a desired location. Since only a boost to the normally much larger permanent magnet tip moment is required, this pulsing strategy may be used but in a selective manner not envisioned in U.S. Pat. No. 6,834,201. Such a strategy can be used to reduce the thermal energy deposited in the coil and its surroundings, and only applied when the navigation controlling computer finds it necessary. If τ is the time constant of catheter mechanical recoil, pulses of longer duration than τ can effectively bend the catheter, thereby allowing larger current spikes in the coil but with an average energy dissipation rate that is acceptable. This heat reduction strategy also can be imposed only when required and as instigated by the navigation controlling computer, for example in response to temperature sensor data.

In specific configurations, an acceptably sized boost coil having a few hundred turns may provide a transverse moment approximately 1/10 the magnitude of a moment of a NdFeB tip magnet.

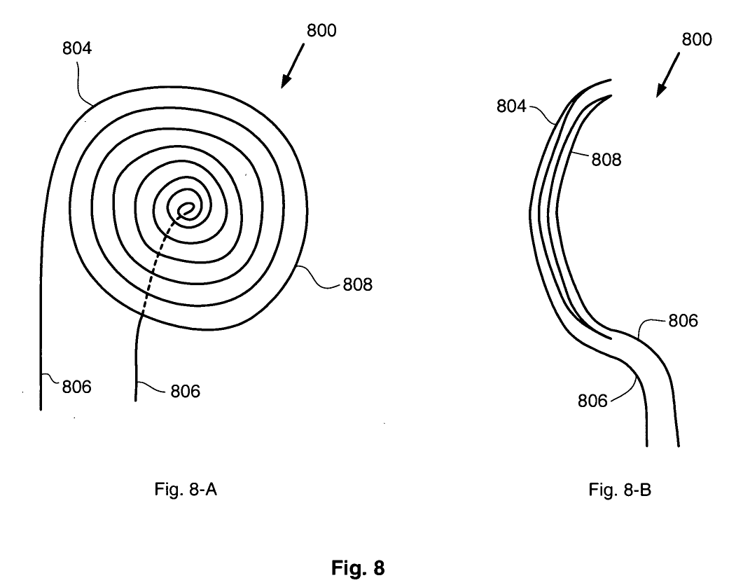

In one exemplary implementation indicated generally in FIGS. 8-A and 8-B by reference number 800, a coil 804 is shown schematically configured as a curved pancake, spanning about ¼ of the circumference of a seed (or tip) magnet having a 2.5-mm diameter. The coil 804 is made, for example, of about 200 turns of AWG 45 wire having a 0.002-inch diameter including insulation and gap. The coil 804 includes leads 806 to a power supply (not shown). Where the wire is coiled, for example, in 5 or 6 layers 808 (one of which is shown in the frontal view of FIG. 8-A and a plurality of which are shown in the edge view of FIG. 8-B), the coil 804 is about 0.012 inches thick. A seed magnet that includes a pair of such coils may not significantly enlarge a catheter tip. For example, a pair of the coils 804 would increase the diameter of a catheter tip by about 0.6 mm. Where a coil 804 has an area of about 0.0045 square inches, two coils 804 together could provide a transverse moment of about 1.17×10^{31 3} I Ampere-m² where I represents current through the coils 804. Where heat dissipation in a coil pair is estimated at about 20 watts, the transverse moment is about 1.4×10⁻³ Ampere-m². For a tip magnet having a magnetization of about 9×10⁵ A/m, a ratio of tip magnetic moment to transverse magnetic moment provided by two coils 804 is about 0.023 to 0.0014, or about 16 to 1.

When a pair of coils 804 dissipates about 20 watts, a gram of fluid (e.g., blood) in the vicinity of the coils 804 would undergo a temperature increase of about 4.8 C. Thus, cooling may not be needed, particularly if the boost was only temporary as is generally the case in applications of systems and methods per this invention. In one configuration wherein cooling is provided, a heat sink (not shown) may be incorporated into the device tip, such as described in U.S. Pat. No. 6,864,201, except that it may be sized with a significantly different strategy as per heat dissipation requirements described below.

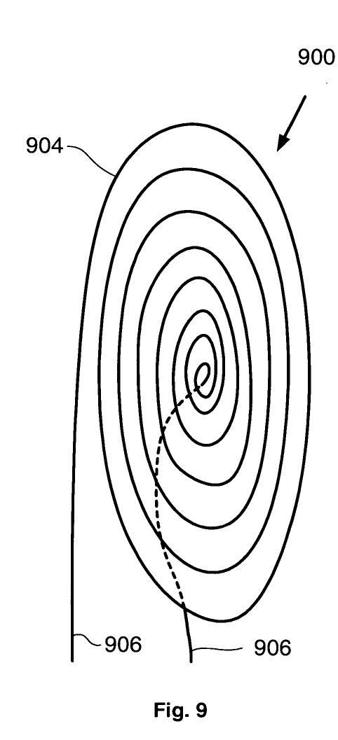

Typically, a tip permanent magnet seed is longer than its diameter. Accordingly, in an exemplary embodiment of a coil indicated generally in FIG. 9 by reference number 900, the coil 904 is elliptical. Providing an elliptical coil makes it possible to provide a larger area, and hence a larger magnetic moment, compared to a circular coil. Coils 904 may be connected in series pairs, each pair having about 400 turns. An elliptical coil 904 having twice the area of a round coil 804 could provide twice the moment provided by the round coil 804. Thus a pair of coils 904 could supply a transverse boost of about ⅛ the axial moment of a tip magnet having a magnetization of about 9×10⁵ A/m.

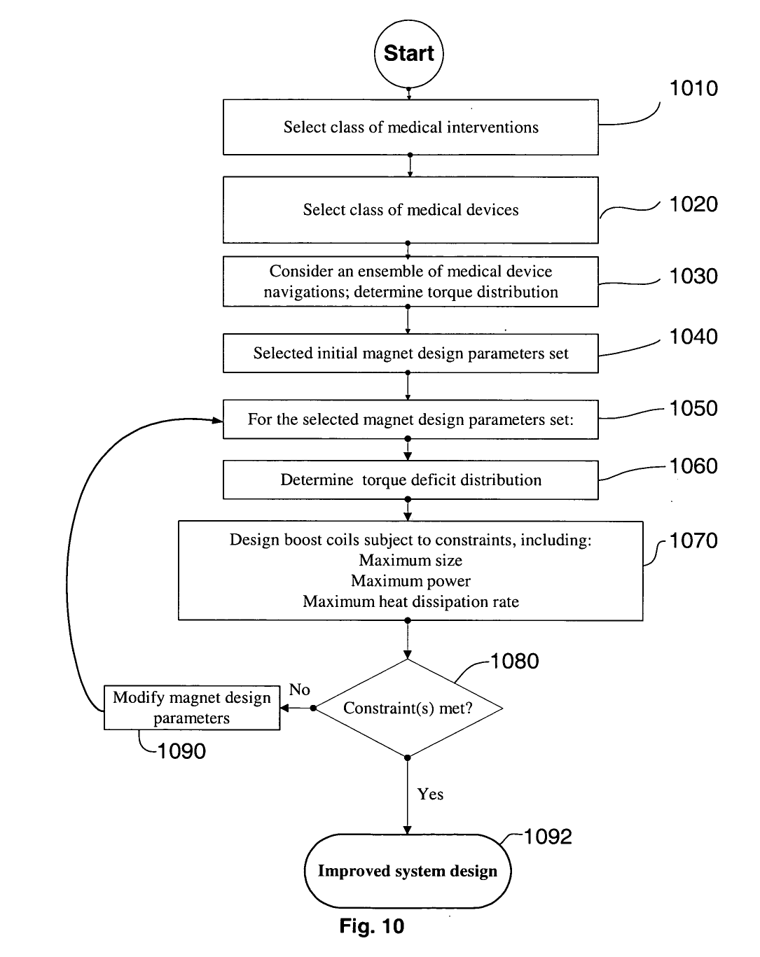

The thermal treatment of the tip coils uses a strategy based upon the total energy needed for a given navigational turn. It has been found, in navigation of a catheter tip of approximately the size described here, that turns may be made in almost any direction and of almost any lead angle for a procedure in which mapping of heart wall signals and subsequent tissue ablation in arrhythmia cases are performed using magnetic navigation. Thus, in the present invention it is assumed that all magnetic field direction changes are possible. A method for improved system design is now described, and illustrated in the flowchart of FIG. 10. In a first step, a class of medical interventions that uses navigation of a medical device (or a set of medical devices) is selected, 1010. Two examples of such classes are (i) treatment of cardiac arrhythmia and (ii) treatment of chronic total occlusions of a coronary artery. In a second step, 1020, a class of medical devices typically used for the selected intervention is defined. In example (i) above, the corresponding class would include cardiac guide wires, sheaths, and ablation catheters. In step 3, 1030, an ensemble or database of case studies for the selected application and devices is analyzed automatically by a computer. A computer algorithm can plot the distribution of catheter bend angles resulting from an ensemble of navigations for a specific class of medical intervention, and the corresponding torque distribution needed. In step 4, 1040, an initial set of magnet design parameters is defined, and selected, step 5, 1050. From this information, the “torque deficit” distribution can be found in step 6, 1060, corresponding to the retained application, medical devices, and selected magnet field source system. This torque deficit distribution will depend on the number and types of source magnets as well as their “size deficit.” The source magnet size deficit is by design, taking into account the capability of a system designed according to the present invention to supply the deficit through boost moments. From this planned deficit the boost coils can be designed, step 7, 1070, taking into account the paired probability of the distribution of angles and of the deficit probability as a function of the angle. The design of the boost coils per the above requirements is subject to a number of intervention-related constraints, including the maximum boost coil size. From these design considerations a second set of specifications is derived, including the maximum power delivered and maximum heat dissipation rate required for a class of intervention and an associated class of medical devices. The design outputs can then be compared to the constraints, box 1080; if one or more of the constraints have been met, while the other parameters are within constraints, then an improved system design 1092 has been found. If one or more of the constraints has been exceeded, it is necessary to iteratively increase the external magnet design parameters, branch 1090; if none of the constraints have been met or exceeded, then there is room to iteratively relax the external magnet design parameters, 1090. Based on design experience and knowledge, familiar to those skilled in the art, transfer functions can be derived that relate the system output parameters to the system design input parameters; such transfer functions can be used to derive optimization algorithms for system design.

Using boost magnets in accordance with principles of the present invention can provide opportunities to increase the performance of a navigation system while reducing its cost. For example, where a source magnet system includes two or more source magnets, a source magnet control algorithm potentially has extra degrees of freedom available, since only three field components are needed at an operating point. These extra degrees of freedom are typically constrained by equations to prevent redundant (i.e., multiple-valued) solutions which otherwise could cause difficulty, such as slow convergence of the navigation algorithm. Such added constraints can be selected to represent desirable system features, for example to provide for uniform or nearly uniform distribution of individual source magnet contributions and/or to speed up system operation. Sizes and locations of boost magnets can be selected to provide more symmetry in source magnet arrangements (such as in their respective positions with respect to the subject) and thereby provide improved guidance in “difficult” operational field directions.

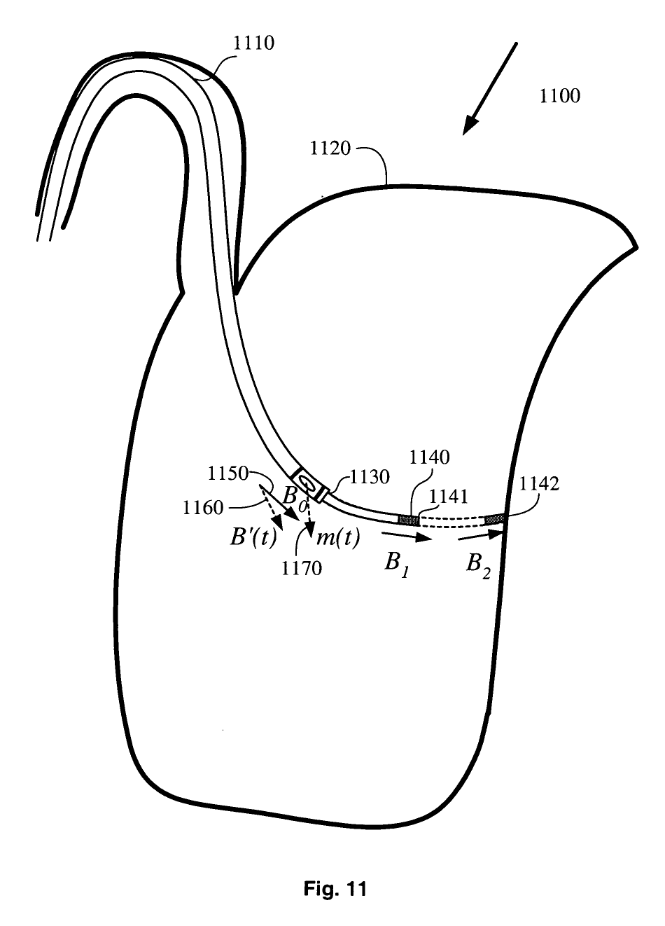

Configurations of the foregoing navigation system and coils can be useful in cardiac mapping. In such procedures, a device tip seed magnet is tilted quickly to the cardiac wall of a subject while a source magnet system supplies a “holding torque” with the device tip away from the cardiac wall. Such procedures currently entail time delays for articulation of large permanent source magnets and/or ramping of large superconducting coil systems. Using configurations of the present system and boost coils can reduce or eliminate such delays, as a holding torque or a tipping torque may be supplied through boost magnets. An embodiment of the present invention with application to cardiac intervention is shown in FIG. 11, 1100. A sheath 1110 has been navigated through the subject's arteries to one of the cardiac chambers 1120. This navigation can be performed, for example, with the catheter magnetic tip advanced to be flush with the sheath distal opening, so as to enable magnetic navigation; alternatively, the sheath and catheter may be advanced over a previously positioned guide wire. The sheath distal end is advanced in the cardiac chamber to position 1130 favorable for subsequent advancement of a catheter tip 1140. The catheter tip is guided to make contact with the cardiac wall at a specific point through a catheter magnetic navigation sequence, two sequence positions being indicated in the figure by 1141 and 1142 (the catheter advanced to position 1142 being shown in dashed lines). During the sequence, it is desirable to maintain the sheath distal end at or near position 1130 and at the orientation indicated by the direction of field B₀, 1150. The magnetic field B′(t) 1160 generated at point 1130 during the catheter navigation sequence will not in general be equal to B₀. This is due to both the navigation field requirements (as indicated by B₁ and B₂ respectively at 1141 and 1142) and to the relative lack of spatial field uniformity (at any given time). However, a computer can calculate B′(t) for the desired catheter navigation sequence and derive accordingly the sequence of boost coil currents to be applied to three boost coils (of three boost coil sets) located at the sheath distal end to generate a compensating moment m(t) 1170 (the moment and local fields are not shown to scale). As an example, and to maintain the sheath in the orientation corresponding to B₀, it is in general desirable to generate a moment m(t) such that B′(t) is at a lead angle to m(t) to create a sheath holding torque; the lead angle between B′(t) and m(t) is calculated by the computer such that the resulting torque counteracts the sheath restraining bending torque. It is clear that such a sequence of holding torques will act to maintain the sheath orientation in the desired direction. In a special situation where the sheath tip would need to be held at a given orientation without bending, it is seen that the moment m(t) would be computer-generated to be aligned with B′(t). Although not shown in the figure, in general the sheath distal end can comprise both a permanent magnetic tip and boost coil sets; similarly the catheter can comprise boost coils sets as well as a permanent magnetic tip.

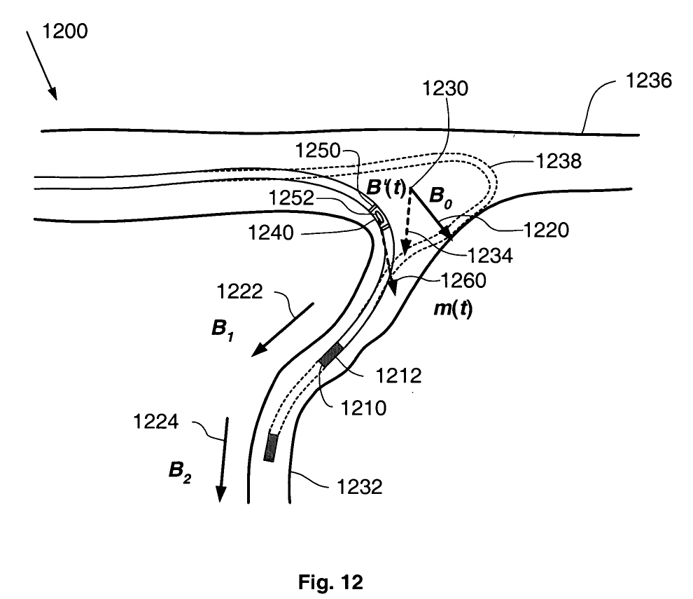

Further, it is clear from the above description of the present inventions that boost magnets may be supplied at any of a number of locations along the length of the medical device. For example, additional magnet pairs may be provided at a distance from the distal end of the medical device to allow generation of a holding torque away from the device tip. This is illustrated in FIG. 12, where a catheter comprising boost coils sets at two locations is shown, 1200. A sequence of magnetic navigation fields is applied at the catheter distal end 1210, which comprises a tip magnet and associated moment 1212. Three instances of the time sequence are shown by B₀ 1220, B₁ 1222, and B₂ 1224. The local magnetic field when the catheter tip was navigated through vessel branch 1230 was B₀, as required to orient the catheter distal end toward lower vessel branch 1232, per the needs of the intervention illustrated. Due to the variation in fields B₁ and B₂ applied as a function of time at the distal end, and due to the spatial variations in the magnetic field (at any time), the field at or near vessel branch 1230 will vary in time through a sequence B′(t) 1234. To prevent the catheter from buckling and being pushed through right vessel branch 1236 (an undesirable situation illustrated by dashed lines 1238), a holding torque can be created at point 1240 along the catheter when that point enters vessel branch 1230. The currents to be dynamically supplied to the boost coils at catheter location 1240 can be calculated as a function of the required “holding” torque to be applied locally to the device, the geometry and mechanical properties of the medical device, and knowledge of the externally applied magnetic field distribution over the operating region as a function of time for the particular magnetic navigation sequence to be applied. Thus, a sequence of boost currents can be applied to the boost coil sets 1250, 1252, and to a third boost coil set not shown in the figure; such boost coil sets defining, for example, three orthogonal axes along which they can generate incremental magnetic moments. The resulting boost moment m(t) then results in a torque being applied on the catheter at position 1240, so that the orientation of that catheter segment can be maintained in a predetermined direction (generally coinciding with the direction of B₀). As an example, and assuming that the field at and near vessel branch 1230 is B′(t) when catheter element at 1240 progresses through the branch, it is desirable to apply boost coil currents such that a local magnetic moment m(t) 1260 is generated such that B′(t) is at a lead angle to m(t); in such a manner, any tendencies of the catheter to buckle and locally reorient will be counteracted by the torque locally applied on catheter element 1240. Depending on catheter stiffness it might be necessary to orient m(t) to increase (or decrease) the local field B′(t) lead angle and resulting torque that counteracts the device restraining mechanical torque. The number of boost coil sets is not limited to two; in other embodiments, such boost coil sets could be distributed along a segment of the medical device so that to enable local control of the device at a number of points.

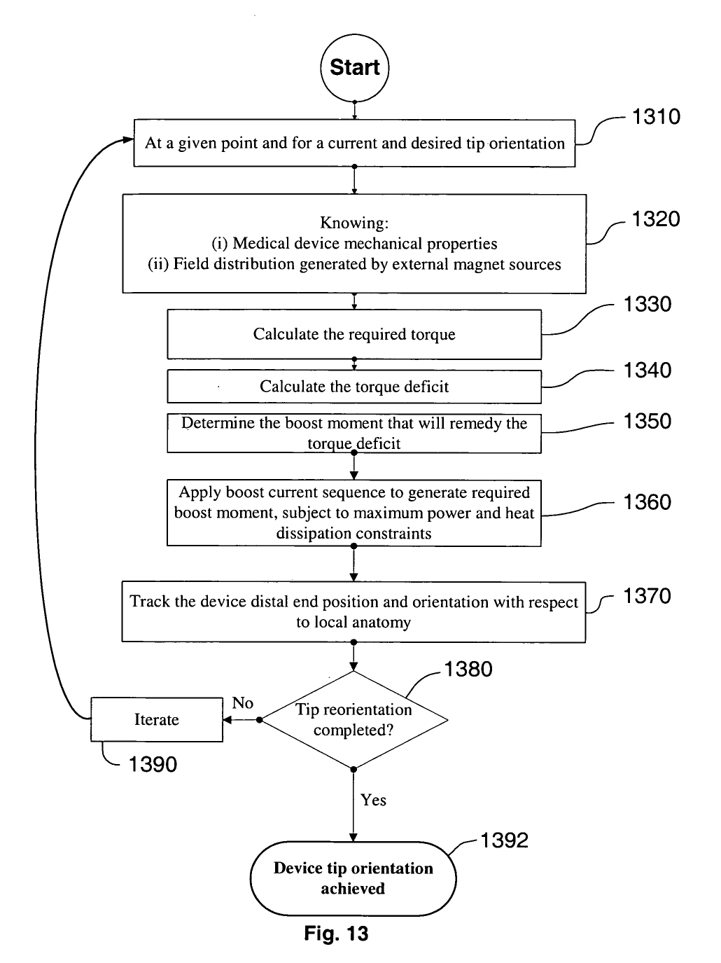

FIG. 13 presents a flow chart of a boost moment generation method according to the present invention. For a given point in the operating volume, and a current and a desired device tip orientation, 1310, and knowing the medical device mechanical properties and field distribution generated by the external magnets, 1320, the required torque to be applied to the device tip is calculated, 1330. From a knowledge of geometry and of the externally applied fields a torque deficit is then derived, 1340. It is then possible to determine the boost moment that will remedy the torque deficit, 1350. The boost currents that will generate the required boost moment are then generated subject to maximum power and heat dissipation constraints, 1360. In certain navigation systems, it is possible to track in real time 1370 the device tip position and orientation with respect to the local anatomy, thus providing a feedback loop to the boost moment determination method, 1380. Such a feedback loop can then be leveraged to dynamically adjust 1390 the boost parameters as may be required for a particular navigation sequence. As a result of the method, the desired device tip orientation is achieved, 1392.

The advantages of the above described apparatus embodiments, improvements, and methods should be readily apparent to one skilled in the art, as to enabling the design of magnetic navigation systems with reduced externally generated magnetic fields; improved navigation of catheters, guide wires, and other related medical devices in a given magnetic navigation system; and faster navigation. Additional design considerations may be incorporated without departing from the spirit and scope of the invention. Accordingly, it is not intended that the invention be limited by the particular embodiment or form described above, but by the appended claims.

1. A method of navigating a medical device in a subject using a magnetic navigation system, the device having a proximal end, a distal end, and an elongated section in between, the distal end having at least one magnetically responsive element with an associated magnetic moment and at least one boost magnet; the magnetic navigation system having at least one source magnet positioned outside the subject generating a magnetic field; the method comprising:
(a) navigating the device tip to a point in the subject, the device tip being oriented in a first direction;
(b) adjusting the at least one source magnet to generate a magnetic field at the device tip of step (a) in a second direction; and
(c) generating a boost magnetic moment using the at least one device boost magnet to increase the torque applied by the magnetic field of step (b) to turn the device tip in a third direction.

2. The method of claim 1, wherein the boost magnetic moment is determined by the navigation system computer.

3. The method of claim 1, wherein generating the boost magnetic moment comprises applying at least one boost current to the at least one boost magnet.

4. The method of claim 3, in which at least one boost magnetic moment can be a negative boost.

5. The method of claim 1, wherein the at least one boost magnet comprises at least one pair of coils adjacent to the at least one device tip magnetically responsive element.

6. The method of claim 3, wherein applying at least one boost current comprises applying a pulsed current.

7. The method of claim 2, further comprising the step of:
calculating the boost moment based on the magnetic field generated at the device tip, the medical device mechanical properties, and the current device tip orientation.

8. A system for magnetic navigation of a medical device in a subject comprising:
(a) a medical device comprising a proximal end, a distal end, and an elongated section in between, the distal end comprising at least one magnetically responsive tip element having associated magnetic moment, and at least one boost magnet;
(b) at least one adjustable source magnet positioned outside the subject;
(c) a source magnet controller;
(d) a boost magnet controller to generate a boost moment by control of the boost magnet(s) of medical device; and
(e) a navigation computer to determine inputs to the source magnet controller and to the boost magnet controller to orient the medical device substantially in a direction.

9. The system of claim 8, wherein the at least one boost magnet comprises three sets of boost coils arranged to be able to provide a moment in any direction.

10. The system of claim 8, wherein two sets of boost coils are positioned attached to the magnetically responsive tip element to be able to provide a moment transverse to the distal end magnetic moment.

11. The system of claim 8, wherein the at least one boost magnet comprises at least one boost coil.

12. The system of claim 11, wherein the at least one boost coil has an elliptical shape.

13. The system of claim 10, wherein the at least one boost coil extends over approximately one fourth of the tip magnet circumference.

14. The system of claim 11, wherein a pair of boost coils is configured to provide a boost moment of at least 0.0014 Ampere meters squared.

15. The system of claim 14, wherein a pair of boost coils is configured to provide a boost moment of at least 0.0003 Ampere meter squared.

16. The system of claim 11, wherein the current to the at least one boost coil is pulsed.

17. The system of claim 16, wherein the pulse parameters are controlled by the navigation computer.

18. The system of claim 8, wherein the at least one source magnet is an articulated permanent magnet.

19. The system of claim 8, wherein the at least one boost magnet is configured to provide a boost magnetic moment to increase the torque applied on the device distal end by the controlled magnetic field by at least 10%.

20.-25. (canceled)

26. A magnetic navigation system for navigating a medical device in an operating region in a subject's body:
an elongate medical device having at least one magnetically responsive element having a magnetic moment, and at least one selectively operable boost element to selectively apply a boost moment, adjacent the distal end;
a magnet system comprising at least one external source magnet for applying a source magnetic field to the operating region, the external source magnets generating a magnetic field strength that creates a sufficient torque with the magnetic moment of the magnetically responsive elements, without the application of a boost moment, to align the distal end of the elongate medical device in most but not all directions in the operating region, and generating a magnetic field strength that creates a sufficient torque with the moment of the magnetically responsive element and the boost moment to align the distal end of the elongate medical device in all directions in the operating region.

27. (canceled)
